(19) United States
(12) Patent Application Publication
(10) Pub. No.: US 2007/0259382 A1
(43) Pub. Date: Nov. 8, 2007

(54) RAPID LOCALIZED CELL TRAPPING ON BIODEGRADABLE POLYMERS

(75) Inventor: ALIASGER K. SALEM, Coralville, IA (US)

Correspondence Address: FULBRIGHT & JAWORSKI L.L.P., 600 CONGRESS AVE., SUITE 2400, AUSTIN, TX, 78701, US

(73) Assignee: THE UNIVERSITY OF IOWA RESEARCH FOUNDATION

(21) Appl. No.: 11/558,540

(22) Filed: Nov. 10, 2006

Related U.S. Application Data
(60) Provisional application No. 60/738,696, filed on Nov. 22, 2005.

(51) Int. Cl. G01N 33/53; B05D 3/00

(52) U.S. Cl. 435/7.5; 427/2.11

(57) ABSTRACT

The present invention provides methods and compositions for cell-based arrays. A biotin-avidin system for the attachment of cells onto a surface also employs biodegradable polymers onto which avidin is disposed in a predetermined pattern.

This application claims benefit of priority to U.S. Provisional Application Ser. No. 60/738,696, filed Nov. 22, 2005, the entire contents of which are hereby incorporated by reference.

The government owns rights in the application pursuant to funding from the National Cancer Institute and Public Health Service Grant number P50 CA097274-04.

### BACKGROUND OF THE INVENTION

I. Field of the Invention

The present invention relates to the fields of cell biology and biochemistry. More particularly, the invention relates to the micropatterning of cells onto surfaces.

II. Related Art

Spatial control over cell binding is essential for modulating cell behavior and engineering cell based sensor arrays (Tien & Chen, 2002; Hyun et al., 2003; Raghavan & Chen, 2004; Patel et al., 1998). Examples of the need for such geometric control over cell binding include neurons that follow adhesion cues to form contacts with muscle and the patterning of fibroblasts and hepatocytes to maximize the cell to cell contact between the two cell types for enhanced viability and functionality over single cell cultures of hepatocytes (Raghavan & Chen, 2004; TessierLavigne & Goodman, 1996; Bhatia et al., 1998). Soft lithography techniques have shown significant potential in facilitating control over the organization of cells (Chen et al., 2005; Kane et al., 1999). Typically, stamps prepared by curing poly dimethylsiloxane (PDMS) over a photolithographic template are used to transfer extracellular matrix (ECM) or cell adhesive molecules directly onto the substrate either by printing or microfluidic networking (Patel et al., 1998; Bhatia et al., 1998; Chen et al., 2005; Kane et al., 1999; Chen et al., 1997; Tan and Desai, 2003; Tourovskia et al., 2005; Patel et al., 2000). To elucidate the properties necessary for optimum cell-substrate interactions, this approach was first used for the transfer of self-assembling molecules onto gold substrates (Mrksich et al., 1997).

More recently, micropatterning of ECM and cell adhesive molecules derived from the ECM (such as RGD from fibronectin) has been carried out on biodegradable substrates (Hyun et al., 2003; Patel et al., 1998; Tan & Desai, 2003; Lin et al., 2005; Liu et al., 2002). Examples of ECMs that are commonly patterned for spatial control over cell attachment include collagen and fibronectin (Bhatia et al., 1998). A limitation to this approach is the need to identify and transfer cell-specific adhesive sequences or ECMs depending on the cell type that is being bound. These methods also remain inapplicable to typically non-adherent cells or cells that do not have sufficient concentrations of functional cell-membrane receptors. In addition, cell (i.e., endothelial) exposure to pulsatile flows after less than a few hours of binding to ECM proteins can result in less than 42% of the cells remaining adhered (Bhat et al., 1998a; 1998b; 1998c; Tsai & Wang, 2005; Miyata et al., 1991). Thus, improved methods for cell micropatterning are needed.

### SUMMARY OF THE INVENTION

In accordance with the present invention, there is provided a method of immobilizing cells comprising (a) providing a surface comprising avidin, wherein in avidin is disposed in a pre-determined pattern; and (b) contacting the surface comprising avidin with a biotinylated cell population, whereby the cell population is immobilized to the surface according to the predetermined pattern. The avidin may be attached to the surface by interaction with a second biotin molecule bound to the surface, and in turn, the second biotin molecule may be bound to the surface through a block copolymer, which is in turn bound to the surface. The block copolymer may be biodegradable. The block copolymer may be silk, elastin, chitin, chitosan, poly(d-hydroxy acid), poly(anhydrides), and poly(athoesters), or more specifically, polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene flumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], poly-hydroxybutyric acid, S-caproic acid, polylactide-co-glycolide, polylactic acid, or polyethylene glycol.

The method may further comprise a step, prior to step (a), of applying the block copolymer to the surface. The method may also further comprise a step, prior to step (a), of applying the avidin to the surface. Applying avidin to the surface may comprise microfluidic networking, such as with a poly-dimethylsiloxane stamp. The method may also further comprise a step, prior to step (b), of biotinylating the cell. The biotinylating of the cell may comprise treating the cell with sodium periodate to generate a cell comprising non-native aldehydes, and reacting the cell comprising non-native aldehydes with biotin-hydrazine.

The surface may further comprise block copolymer lacking avidin in areas outside the predetermined pattern. The block copolymer lacking avidin may be biodegradable. The block copolymer lacking aviding may be the same or different as compared to the block copolymer comprising avidin. The cell population may be bound to the predetermined pattern at levels about 10-fold, about 15-fold, about 20-fold or about 25-fold higher than outside the predetermined pattern. The cell population may be naturally adherent or naturally non-adherent. The cell population may be a fibroblast population, an endothelial cell population, a neuronal cell population, or an epithelial cell population. The surface may comprise silicon, plastic, glass or paper. The surface may be disposed in a culture dish, a biochip, a column, or a filter. The predetermined pattern may comprise cells dispersed in a line, a square, a rectangle, a circle, an oval or combinations thereof. The cell population, when bound to the surface, may be 50% viable, 55% viable, 60% viable, 65% viable, 70% viable, 75% viable, 80% viable or 85% viable.

In another embodiment, there is provided a device comprising (a) a surface; (b) a biotinylated copolymer bound to the surface; (c) avidin bound to the biotinylated copolymer in a predetermined pattern; and (d) a biotinylated cell population bound to the avidin. The biotinylated copolymer may be biodegradable. The block copolymer may be silk, elastin, chitin, chitosan, poly(d-hydroxy acid), poly(anhydrides), and poly(athoesters), more specifically, polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], poly-hydroxybutyric acid, S-caproic acid, polylactide-co-glycolide, polylactic acid, or polyethylene glycol. The surface may comprise silicon, plastic, glass or paper. The device may be a biosensor. The predetermined pattern may comprise cells dispersed in a line, a square, a rectangle, a circle, an oval or combinations thereof. The cell population may be a fibroblast population, an endothelial cell population, a neuronal cell population, or an epithelial cell population.

In yet another embodiment, there is provided a method of screening an agent for an effect on a cell population comprising (a) contacting the agent with a device comprising (i) a surface; (ii) a biotinylated copolymer bound to the surface; (iii) avidin bound to the biotinylated copolymer in a predetermined pattern; and (iv) a biotinylated cell population bound to the avidin; and (b) assessing the effect of the agent on the cell population. The agent may be a peptide, a protein, an organopharmaceutical, a lipid, a carbohydrate or a nucleic acid.

It is contemplated that any method or composition described herein can be implemented with respect to any other method or composition described herein.

Throughout this application, the term “about” is used to indicate that a value includes the standard deviation of error for the device or method being employed to determine the value. In particular, about is defined as +/−5% of the stated value.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1—Schematic of preparation of spatially controlled biotinylated cells on biodegradable templates. Step a) Biotin is covalently attached to α-hydroxy-w-amine PEG. Step b) Lactide is graft polymerized onto hydroxyl terminus of biotin-PEG-OH. Step c) PLA-PEG-biotin is formed into a thin film. Step d) SU8 photoresist is cast over a silicon wafer and exposed to UV through a patterned master. Step e) A PDMS mold is formed over the patterned template which, then Step f) forms a seal with the PLA-PEG-biotin film. Step g) Avidin that is flowed through the microfluidic channels becomes immobilized in the predefined channel regions. Step h) HDF cells are treated with sodium periodate to convert native sialic residues into non-native aldehyde groups. Step i) The aldehyde groups on the surface of the cells are reacted with biotin hydrazide to produce biotinylated cells which are, then Step k) incubated on the avidin patterned PLA-PEG-biotin template to produce the patterned cells.

FIGS. 2A-F—(FIG. 2A) Fluorescent microscopy image (Olympus BX40, 555/580 nm) showing celltracker red stained avidin treated biotinylated HDF cells with corresponding images (FIG. 2B) showing a fluorescent microscopy image of hoescht (350/450 nm) staining and (FIG. 2C) fluorescence emanating from the FITC-avidin (494/518 nm) bound to the biotinylated HDF cells. (FIG. 2D) A bar chart comparing cell attachment of biotinylated cells on avidinylated PLA-PEG-biotin substrates, PLA-PEG-biotin substrates and TCP with n=9. (FIG. 2E) A fluorescent microscopy image of the patterned tetramethylrhodamine-avidin (av-rh, 555/580 nm) immobilized on the PLA-PEG-biotin substrate. (FIG. 2F) Biotinylated cells stained with celltracker green (494/518 nm) bound specifically within the av-rh (555/580 nm) patterned channels.

### DETAILED DESCRIPTION OF THE PREFERRED EMBODIMENTS

I. The Present Invention

The inventors now describe a biotin-avidin based patterning procedure that can be utilized for facile spatially controlled rapid attachment of a wide range of cell types. This is achieved by converting native sialic residues on the surface of cells into non-native aldehydes using a mild sodium periodate treatment. Sialic acids are a common terminal cell surface monosaccharide group with increased expression in many cancers (Prescher et al., 2004; Luchansky et al., 2004). Cell surface modification has been utilized to engineer cell-cell interactions between myoblasts and to bind endothelial cells, mouse ascite carcinoma cells and chondrocytes to glass or tissue culture plastic substrates with high attachment efficiencies (Bhat et al., 1998b; 1998c; Tsia & Wang, 2005; De Bank et al., 2003). The periodate reaction produces a significant proportion of reactive aldehyde groups (De Bank et al., 2003). The aldehyde groups are then reacted with biotin-hydrazide to produce biotinylated cells. Avidin is patterned onto the surface of a biotinylated biodegradable block copolymer, polylactide-poly (ethylene glycol)-biotin (PLA-PEG-biotin) (Salem et al., 2004), for example, by microfluidic networking using a PDMS stamp (Salem et al., 2004). The biotinylated cells then bind specifically to the patterned avidin regions. The PEG that is presented from the PLA-PEG-biotin copolymer without avidin immobilization minimizes cell binding in the non-patterned regions (Salem et al., 2001; Salem et al., 2003). These and other details of the invention are described below.

II. Cell Types

Virtually any cell type and size can be attached to surfaces in accordance with the present invention. The cells may be prokaryotic or eukaryotic, plant, fungal, animal or human. The cells may be neuronal, endothelial or epithelial in origin, or may be lymphocytes, fibroblasts or myoblasts. Particular cells types include bacteria such as E. coli, Staphylococcus, myoblast precursors to skeletal muscle cells, neutrophils, erythroblast, osteoblasts, chondrocyte cartilage cells, basophil, eosinophils, adipocyte fat cells, invertebrate neurons (Helix aspera), mammalian neurons, adrenomedullary cells, melanocytes, or cancer cells. In addition, naturally occurring or genetically engineered cells, including those form plants, mammals, or invertebrates may be employed as well as mixtures of cells. A particularly useful source of cell lines may be found in ATCC Cell Lines and Hybridomas (8th ed., 1994), Bacteria and Bacteriophages (19th ed., 1996), Yeast (1995), Mycology and Botany (19th ed., 1996), and Protists: Algae and Protozoa (18th ed., 1993), each of which are herein incorporated by reference.

III. Degradable Polymers

The present invention contemplates the use of biocompatible polymers, including biodegradable polymers, as the vehicle to link avidin, and indirectly cells, to a substrate. The biocompatible polymer may comprise silk, elastin, chitin, chitosan, poly(d-hydroxy acid), poly(anhydrides), and poly(athoesters). More particularly, the biocompatible polymer may comprises polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], poly-hydroxybutyric acid, or S-caproic acid, polylactide-co-glycolide, polylactic acid, and polyethylene glycol.

IV. Surfaces

A variety of different surfaces may be coated with polymers according to the present invention, and further, patterned with avidin using techniques discussed below. In particular, it is envision that surface made of silicon, a variety of different plastics and polymers, glass or paper maybe used. These surfaces may be part of a culture dish or plate, including multiwells, a bead, a fiber, a biochip, a column, a dipstick or a filter.

V. Methods of Localizing Cells

The present invention relies on the use of a biotin-avid system to pattern cells onto a substrate. Though biotin-avidin is the exemplified form, other ligand-binding partner pairs may be utilized.

The general approach is to derivatize cells such that they accept a first binding partner on their cell surface in a reversible or non-reversible fashion. Cells are then attached to a surface that has been coated with a biodegradable polymer, onto which the second binding partner has been patterned. Finally, one contacts the patterned surface with the derivatized cells carrying the first binding partner, followed by a wash step to remove non-specifically bound cells.

A. Protocol for Surface Functionalization of Cells with Biotin

An examplary protocol for surface functionalization of cells with biotin is provided below. First, the following reagents must be prepared:

Biotin Buffer:
Phosphate buffered saline (PBS)
0.1% FBS
pH 6.5
Biotin hydrazide solution:
5 mM in biotin buffer
FITC-avidin solution
500 μg/mL in distilled water (cover vial w/Al foil)
Sodium Periodate solution:
1 mM in phosphate buffered saline
Avidin buffer:
Phosphate buffered saline
0.1% FBS
pH 7.4
FITC-avidin solution:
5 μg/mL in avidin buffer (cover w/Al foil)
Hoescht 33258 (Bisbenzimide)
0.01 mg/ml in phosphate buffered saline (cover vial with Al foil)

Cells are grown to 80-90% confluence. Next, Cell Tracker Red fluorescent stain is prepared by reconstituting the material in 11 μl dimethylsiloxane (DMSO), which is then added to warmed media. The cells are incubated with media containing the fluorscent stain for 45 mins in the incubator, after which the media is replaced with fresh warmed media and incubate for a further 1 hr. Cells are washed twice with PBS at room temperature, and then incubated with 1 mM solution of sodium periodate in cold PBS for 15 mins in the dark at 4° C. Cells are washed twice with biotin buffer at room temperature. Next, the cells are incubated with biotin hydrazide solution (5 mM in biotin buffer) for 90 min at room temperature. The cells are washed twice with avidin buffer twice and then incubated with the FITC avidin solution at 4° C. for 15-20 mins with FITC-avidin.

For top quality images, prepare one set of cells to which Hoescht 33258 in PBS is added for 15 mins at 4° C. in the dark. This cell set is washed with avidin buffer twice at room temperature. All cell sets are washed two times with cold avidin buffer, and the cells are imaged live under a microscope using all three different fluorescent channels, and then pictures are then merged. As controls, one set of cells is utilized where all the steps are performed except for periodate functionalization, and another set where all steps except are performed expect for the biotin hydrazide reaction. Also, one cell set is used to determine cell viability using trypan blue measurement.

B. Patterning of Avidin on Block Copolymers

The present invention employs microfluidic processes to apply avidin, or another binding partner, onto a surface. Microfluidics refers to a set of technologies that control the flow of minute amounts of liquids or gases, typically measured in nano- and picoliters, in a miniaturized system. Although micro- and macrofluidics systems require similar components, including pumps, valves, mixers, filters, and separators, the small size of microchannels causes their flow to behave differently. Hence, microscale components require new fabrication methods.

Microfluidics devices, first developed in the early 1990's, were initially fabricated in silicon and glass using photolithography and etching techniques adapted from the microelectronics industry, which are precise but expensive and inflexible. The trend recently has moved toward the application of soft lithography, i.e., fabrication methods based on printing and molding organic materials, to build microdevices. These techniques enable the construction of three-dimensional networks of channels and components, and they provide a high level of control over the molecular structure of channel surfaces.

Microfluidics' appeal lies in the fact that the microchips require only a small amount of sample and reagent for each process—only a few tens or hundreds of nanoliters compared with the 100 ml required by existing plate assays. Microscale reactions also occur much faster because of the unique physics of small fluid volumes, and microfluidics technologies are easily automated to do routine assay and sample preparation on standardized chips with little human intervention. Such chips hold the promise of combining multiple functions on a single chip, including purification, labeling, reaction, separation, and detection. Microfluidics would guide the sample automatically from one station to another on the chip.

C. Protocol for Assessing Attachment of Biotin Functionalized Cells in Spatially Defined Regions

The following protocol is an examplary and non-limiting approach to assessing attachment of biotin Functionalized cells to prepared surfaces.

The PDMS stamp is prepared by curing 9:1 siloxane monomer with a curing agent at 60° C. over a lithographic template overnight. PLA-PEG-biotin is spin cast onto plastic at 1 mg/ml in TFE, and the TFE is allowed to evaporate overnight under a fume hood. The stamp is cut out (very carefully) from the lithographic template. The PDMS stamp is washed repeatedly with ethanol, hexane and d.i. water cycles using lens cleaning tissue. The stamp is dried using air. Using visual judgement, the best seal possible is created the stamp and the PLA-PEG-biotin polymer substrate. FITC-avidin solution is added as a droplet positioned so that the FITC-avidin solution wets an end of the mold where the capillaries are open. After 1 hr of contact, the remaining FITC-avidin is removed by blotting and replaced with 5 ml of distilled water. After 5 min, the water is removed and the washing procedure is repeated 5 times. The sample is immersed in water and the mold is removed by carefully peeling apart the PLA-PEG-biotin substrate and the PDMS. The sample is washed with an additional 50 ml of water three times stored in a covered dish.

Cells are grown to 80-90% confluence. Next, Cell Tracker Red fluorescent stain is prepared by reconstituting the material in 11 IIl dimethylsiloxane (DMSO), which is then added to warmed media. The cells are incubated with media containing the fluorscent stain for 45 mins in the incubator, after which the media is replaced with fresh warmed media and incubate for a further 1 hr. Cells are washed twice with PBS at room temperature, and then incubated with 1 mM solution of sodium periodate in cold PBS for 15 mins in the dark at 4° C. Cells are washed twice with biotin buffer at room temperature. Next, the cells are incubated with biotin hydrazide solution (5 mM in biotin buffer) for 90 min at room temperature. The cells are trypsinized and neutralized with neutralizing medium while cells are in suspension. The cells are gently mixed in warmed medium and incubate with patterned FITC-avidin substrates. The media/cells are gently swirled around the dish to get as best a distribution of cells as possible, and then are incubated in the dark at 4° C. for 10 min. Any excess media is removed along with any unbound cells, keeping the media for the moment. After confirming that cells are bound (and if not, then repeat using the retained cells/media), wash the substrate twice using warmed avidin buffer (gently, introducing the buffer from the side and removing from the side so as not to dislodge any bound cells).

For top quality images, prepare one set of cells to which Hoescht 33258 in PBS is added for 15 mins at 4° C. in the dark. This cell set is washed with avidin buffer twice at room temperature. All cell sets are washed two times with cold avidin buffer, and the cells are imaged live under a microscope using all three different fluorescent channels, and then pictures are then merged. As controls, one set of cells is utilized where all the steps are performed except for periodate functionalization, and another set where all steps except are performed expect for the biotin hydrazide reaction. Also, one cell set is used to determine cell viability using trypan blue measurement.

D. Assessing Cell Viability

Following application to a surface, it may be desireable to assess the viability of the patterned cells. Cell viability may be investigated using a pH indicator, 2′-7′-bis-(2carboxyethyl)-5-(and-6-)-carboxyfluorescein (BCECF-AM) which has an excitation wavelength of 505 nm and an emission wavelength of 535 nm and is available from Molecular Probes (Eugene, Oreg.). The acetoxymethyl (AM) ester form of BCECF is non-fluorescent in solution. The BCECF-AM is cell membrame permeant and passively enters the cell where, once inside the cell, the lipophilic blocking groups are cleaved by non-specific esterases resulting in an increase in fluorescent intensity. This increase in fluorescent intensity is indicative of the cell viability.

In a alternative embodiment, BCECF-Dextran, available from Molecular Probes, can be used for cell viability measurements. A 0.1 μM solution of the dye is added to cell media contained within array microwells. BCECF requires excitation at two wavelengths, 490 nm and 440 nm, and the ratio of the emitted light intensity at 530 nm for each wavelength is proportional to pH. This dye is conjugated with a large Dextran group to prevent entry into the cell through the cell membrane. Thus, BCECF-Dextran can monitor decreases in pH within the external cell environment due to cell metabolism.

In yet another embodiment, a commercial cell viability assay, LIVE/DEAD from Invitrogen may be employed. This assay provides a two-color fluorescence cell viability assay based on intracellular esterase activity and plasma membrane integrity. Live cells are distinguished by the enzymatic conversion of the cell-permeant non-fluorescent calcein AM to fluorescent calcein, with an excitation wavelength at 495 nm and an emission wavelength at 515 nm. Dead cells are distinguished by binding ethidium homodimer (EthD-1), with an excitation wavelength at 495 nm and an emission wavelength at 635 nm, to nucleic acids which is accompanied by a 40-fold increase in fluorescent intensity. EthD-1 is excluded by the intact plasma membranes of living cells. Background fluorescence levels are inherently low with this assay technique because the dyes are virtually non-fluorescent before interacting with cells.

Another assay is Promega's CellTiter-Blue™ Cell viability assays. This easily automated assay is flexible and allows you many choices between fluorometric or colorimetric detection methods, 96- or 384-well formats, opportunity to multiplex assays, and choice of the duration of incubation to maximize sensitivity. The assay procedure involves addition of a single reagent (CellTiter-Blue™ Reagent) directly to cells cultured in 96- or 384-multiwell plates, incubation for 1-4 hours, and recording fluorescence or absorbance. The signal produced by conversion of resazurin to resorufin is directly proportional to viable cell number.

VI. Biosensors and Cell Microarrays

A. Biosensors

A biosensor is an analytical device which converts a biological response into an electrical signal. The term “biosensor” is often used to cover sensor devices used in order to determine the concentration of substances and other parameters of biological interest even where they do not utilise a biological system directly. This very broad definition is used by some scientific journals (e.g., Biosensors, Elsevier Applied Science) but will not be applied to the coverage here.

The biological response of the biosensor is determined by the biocatalytic membrane which accomplishes the conversion of reactant to product. In accordance with the present invention, the membrane is comprised of cells patterned on the the biosensor's surface. Such as biosensor can be applied to a large variety of conventional assays for screening and detection purposes. The biosensor may be configured for virtually any assay and offers a distinct advantage for high throughput screening where a plurality of encoded cell populations, which have utility in particular assays or are genetically engineered cell to provide unique responses to analytes, may be employed in a single sensor array for conducting a large number of assays simultaneously on a small sample. The biosensor array thus provides both for tremendous efficiencies in screening large combinatorial libraries and allows conduction of a large number of assays on extremely small sample volumes, such as biologically important molecules synthesized on micron sized beads. The biosensor of the present invention can be applied to virtually any analyte measurements where there is a detectable cell response to the analyte due to biological stimulation.

The biosensor array and methods of the present invention utilizes the unique ability of living cell populations to respond to biologically significant compounds in a characteristic and detectable manner. Since the selectivity of living cells for such compounds has considerable value and utility in drug screening and analysis of complex biological fluids, a biosensor which makes use of the unique characteristics of living cell populations offers distinct advantages in high throughput screening of combinatorial libraries where hundreds of thousands of candidate pharmaceutical compounds must be evaluated. In addition, such a biosensor and sensing method can be utilized for either off-line monitoring of bioprocesses or in situ monitoring of environmental pollutants where the enhanced sensitivity of living cells to their local environment can be exploited.

Sensors of chemical and biological agents, including viral and bacterial pathogens, are important to clinical diagnostics, food monitoring, and detection of bio-warfare agents in urban and military settings. Cells and tissues have several characteristics that make them better suited for sensing these targets than isolated molecules. For example, cells present multiple receptors (some of which have low specificity for single targets) and rely on complex non-linear information processing that allows them to identify agents with high accuracy. Cells also employ amplification schemes to improve sensitivity and reduce response times. The use of cells as sensor elements still requires that the cells be joined with a materials device and that the natural transduction mechanisms of living cells be translated to give electrical outputs from the device.

While the examples below provide a variety of specific assays which may be useful in configuring and employing the biosensor array and method of the present invention, they are not intended to limit either the scope of applications envisioned or the broad range of sensing methods which can be employed with a plurality of cell populations with the biosensor of the present invention.

In one embodiment, the biosensor array can be employed for remotely monitoring redox states of individual cells or cell populations in bioprocesses. For example, NADH dependent fluorescence can be measured in bacteria, fungi, plant or animal cells. NAD(P)/NAD(P)H can be measured to monitor changes from aerobic to anaerobic metabolism in fermentation processes using the method disclosed by Luong et al. (1990).

Alternatively, the biosensor array may be employed for in situ monitoring of cellular processes in response to environmental contaminants by incorporating the method disclosed by Hughes et al. (1995) to provide for distinguishable cell population responses within an array. In this method, micron-sized spheres, impregnated with a fluorophore and modified on the surface with a fluorogenic enzyme probe, are ingested by cells and enzymatic activity occurs at the sphere surface, producing a detectable fluorescent signal.

In yet another embodiment, the biosensor array can be employed with genetically engineered bioluminescent bacteria for in situ monitoring and optical sensing of metallic compounds. For example, cell population responses to antimonite and arsenite may be utilized by incorporating the method disclosed in Ramanathan et al. (1997) into cell populations within the biosensor array. With this method, cell plasmid regulates the expression of bacterial luciferase depending on the metal concentration.

In another embodiment, the cell populations within the biosensor array can be transformed with ATP-dependent luminescent proteins, for example firefly luciferase, which are injected into rat hepatocytes for pathological studies according to the method disclosed by Koop et al. (1993). These cells exhibit a decrease in cytoplasmic ATP when exposed to pathological insults and changes in fluorescence directly relate to the extent of metabolic poisoning in the cell.

In one embodiment, the cell populations within the biosensor array can be transformed with green fluorescent protein (Gura, 1997; Niswender et al., 1995; Cubitt et al., 1995; Miyawaki et al., 1997). Several genetically-engineered mutants of GFP are available which have distinguishable fluorescence emission wavelengths. These proteins have additional utility as fluorescing indicators of gene expression and calcium levels within cells.

In an additional embodiment, the biosensor array can be used in measurements of cell proliferation by in situ monitoring of calcium levels and calcium oscillations in single cells using fluorescent markers, such as aequorin or fura-2, according to the method disclosed by Cobbold et al. (1990).

Electrical transduction of cell-based sensor phenomena includes impedance imaging of cell motility at the nm level and extracellular potentials from electrically excitable tissue, in particular those of neuronal and cardiac origin. Networks of excitable cells cultured on microelectrode arrays are uniquely poised to provide rapid, functional classification of an analyte and ultimately constitute a potentially effective cell-based sensor technology. In recent years, the use of microfabricated extracellular electrodes to monitor electrical activity in cells has gained increasing use. Planar arrays have been used to record extracellular potentials from tissue slices including the vertebrate retina and hippocampal organotypic slices, although longevity of the slice preparation is a major concern. In addition, extracellular recordings have been performed from dissociated mammalian neurons from superior cervical ganglia, mouse dorsal root ganglia, and spinal cord.

The most prominent work in electrical transduction for cell-based sensor applications has been performed by Gross and colleagues at the University of North Texas. In fact, their precise methodological approach that generates a co-culture of glial support cells and randomly seeded neurons resulting in spontaneous bioelectrical activity ranging from stochastic neuronal spiking to organized bursting and long-term oscillatory activity. Surveys of neurotransmitters and neurotoxins indicate that modulation of electrophysiologic parameters, such as extracellular action potential amplitude and burst rate, are indicative of compounds that appear to be functionally distinguishable as excitatory, inhibitory, disinhibitory, or cytotoxic.

One approach uses microelectrode arrays to monitor ion channel activity in adherent neuronal cells. This strategy is well-suited for detecting neurotoxins and other chemical agents that act against membrane channel receptors. Several research groups have developed and fabricated integrated arrays that are tailored to these applications and have developed microfluidic cassettes that permit automated sample introduction and assays. There have also been important advances in developing pattern recognition systems that can identify with better accuracy the source of changes in electrical activity.

Another approach has used cells that are engineered to give spectroscopic signals in response to specific signal transduction pathways. Most strategies use cells that are transfected with green fluorescent protein, and can take many forms. Cells that are engineered to express the GFP under the control of specific promoters report on the promoter activity. In other strategies, cells are engineered such that GFP fusion proteins undergo translocation within the cell; for example, localization of transcription factors from the cytosol to the nucleus. Other strategies rely on fluorescence energy transfer between pairs of chromophores. This class of cell-based sensors offers wide flexibility in engineering cells to respond to a range of targets because they give direct information on key molecular processes within the cell. There have also been important advances in developing software architectures for storing and mining fluorescence data in order to give robust identification of targets.

A variety of biosensors have been described in the patent literature, and following are incorporated by reference in their entirety: U.S. Pat. Nos. 6,856,125, 6,667,159, 6,605,039, 6,603,548, 6,577,780, 6,544,393, 6,377,721, 6,329,160, 6,274,345, 6,258,254, 6,210,910, and 5,177,012.

B. Microarrays

Cell-based microarrays were first described by Ziauddin and Sabatini in 2001 as a novel method for performing high-throughput screens of gene function. In that study, expression vectors containing the open reading frame of human genes were printed onto glass microscope slides to form a microarray. Transfection reagents were added pre- or post-spotting, and cells were grown over the surface of the array. The authors demonstrated that cells growing in the immediate vicinity of the expression vectors underwent “reverse transfection,” and that subsequent alterations in cell function could then be detected by secondary assays performed on the array. Subsequent publications have adapted the technique to a variety of applications, and have also shown that the approach works when arrays are fabricated using short interfering RNAs and compounds.

The advantage of cell microarrays, as opposed to isolated protein/carbohydrate/lipid arrays/nucleic acid arrays is that the activity of the functioning cell can be assessed in toto. However, they retain the advantage of minaturization, high throughput and relatively low cost as compared to cell culture methods. However, mammalian cell finction is highly sensitive to external stresses and surface morphology, and it has been shown that decreasing cell size results in an increase in protein secretion whereas cell growth decreases. Also, cell life and death has been shown to be dependent on surface geometry, implying that forcing cells into different pre-defined surfaces geometries can give crucial insight into cell function and behavior.

In the case of technological applications, a wide range of sensors are already being developed. Examples include: piezoelectric cell-based biosensors for physiological investigations, microelectrode array biosensors for detection of extracellular potentials, pathogen identification biosensors for, e.g., diagnostics of infectious diseases,₁₂ nanocalorimetric biosensorsl₁₃ and immunosensors for detection of clinical analytes. Living cell biosensors may also be applied for detection of, e.g., biological agents or biohazards.

VII. Screening Methods

The present invention provides methods for screening of agents for their effect on a cell population affixed to a surface in accordance with the present invention. The drugs may be known therapeutics, may be members of large libraries of candidate substances, or may be specific compounds assessed for particular effects. The functions examined include binding to, uptake by, or accumulation in the cells. Other assays may look at biological effects on the cells as a whole, such as transport, respiration, viability, while others may assess the expression of a target protein, or function of particular molecules within the cells, such as proteases, kinases, phosphatases, glycosylases, lipases, endonucleases, helicases, polymerases, transcriptases, exonucleases, ATPases, or peptidases.

It will, of course, be understood that all the screening methods of the present invention are useful in themselves notwithstanding the fact that effective candidates may not be found. The invention provides methods for screening for such candidates, not solely methods of finding them.

In particular embodiments, the present invention provides a method for screening agents in test tubes, plates, dishes, or on fibers, filters dipsticks, beads or other suitable surfaces. Various cells and cell lines can be utilized for screening assays following attachment to a surface, and these are discussed elsewhere.

For candidate substances, one may use known drugs or candidates with known or suspected activities. On the other hand, one may simply acquire, from various commercial sources, small molecule libraries that are believed to meet the basic criteria for useful drugs. Screening of such libraries, including combinatorially generated libraries (e.g., peptide libraries), is a rapid and efficient way to screen large number of related (and unrelated) compounds for activity. Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. Other suitable modulators include lipids, sugars, peptides, proteins (enzymes, antibodies), or nucleic acids (antisense molecules, ribozymes, siRNA). Such compounds are described in greater detail elsewhere in this document.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials & Methods

PLA-PEG-Biotin synthesis. α-hydroxy-w-amine PEG (1 g) was dissolved into acetonitrile (2 ml, Aldrich), methylene chloride (1 ml, Aldrich) and Et₃N (80 ml, Aldrich). After addition of NHS-Biotin (0.250 g, Sigma), the reactants were stirred overnight under argon. The reaction was worked-up by the slow addition of diethyl ether (40 ml, Aldrich) to precipitate the polymer. The polymer was re-precipitated from hot isopropanol (70° C., Aldrich). The polymer (350 mg) was dried azeotropically and left under vacuum. Lactide (2 g, Purac biochem bv) was added to biotin-PEG-OH (0.35 g) and diluted with 10 ml toluene, Sn(Oct)₂/toluene (0.1 g in 1 ml). The reaction was then brought to reflux at 110° C. for 4 hours under argon. The product was precipitated from a dichloromethane solution into a cold stirring solution of diethyl ether and isolated by vacuum filtration. Final product was assessed by gel permeation chromatography (GPC) and ¹H-NMR spectroscopy.

PDMS stamp preparation. To fabricate the mold, a 9:1 ratio of siloxane monomer (Sylgard Silicone Elastomer 184, Dow Corning) to curing agent was cured overnight at 50° C. on a patterned master. The master was prepared by spin coating 250 μl photoresist (SU8) onto a silicone wafer for 55 s at 2500 rpm, solvent-baked at 100° C. for 100 s, and then exposed to UV light (11 mJ cm⁻²) from a mercury vapor lamp. The exposed resist was developed in a 4:1 mixture of deionized water and dried with nitrogen. The patterned master was then hard-baked for 25 min at 125° C. The elastomeric mold with the negative imprint on it was peeled off and washed several times with ethanol, hexane, and deionized water.

Cell culture. Human Dermal Fibroblasts (HDF, Cambrex) were maintained in Dulbecco's modified Eagle's medium (DMEM, Gibco) supplemented with 10% bovine calf serum, 0.5% penicillin, 0.5% streptomycin and 1% L-glutamine in a humidified incubator at 37° C. and 5% CO₂. Cells were passaged 1:4 every 5 days before reaching confluence. Fresh media was added every 2-3 days.

Fluorescence Spectroscopy Studies. Using the [2-(4′Hydroxyazobenzene)benzoic acid] (HABA)/Avidin Reagent, spectroscopy studies were completed on a SPECTRAmax PLUS (Molecular Devices) at a fixed wavelength of 500 nm at 37° C. The average read time was 0.5 s and the read mode [Abs]. Three blanks were read followed by three readings of the HABA/avidin in 1 ml cuvettes. Each sample was recorded three times over a subset of four repeats. The assay was completed by measuring the absorbance of the avidin-HABA complex at 500 nm before and after (10 mins) it had been placed over a monolayer of biotinylated cells cultured in 6-well plates. The absorption decreases proportionately to the biotin present on the surface because biotin displaces HABA due to its higher affinity for avidin. The change in absorbance can then be used to calculate the amount of biotin present. A series of biotin solutions of varying concentration were prepared as a calibration curve for determining the quantity of biotin molecules present of the cell surface.

### EXAMPLE 2

### Results

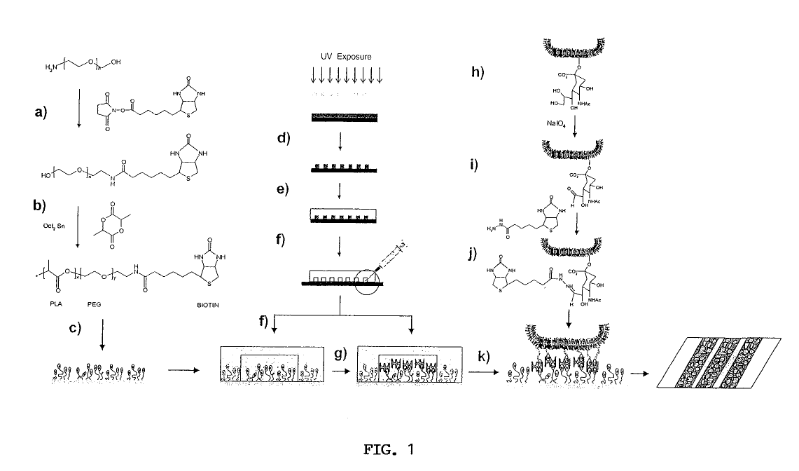

FIG. 1 shows schematically the approach for patterning of cells using the biotin-avidin interaction. First, a biotinylated poly lactic acid-polyethylene glycol copolymer is synthesized by reacting N-hydroxysuccinimide-biotin with the amine terminus of a bifunctional α-amine-ω-hydroxy-polyethylene glycol that was prepared by reducing α-amine-ω-carboxylic acid-polyethylene glycol (Nektar Therapeutics) in a 1M tetrahydrofuran-Borane mixture (Sigma). Confirmation of the amide bond between the biotin and the PEG was observed by the appearance of a triplet at 7.8 ppm in ¹H-NMR. Lactide (Purac Biochem bv) was then graft polymerized onto the hydroxyl terminus of the α-biotin-ω-hydroxy-polyethylene glycol in the presence of a stannous 2 ethyl hexanoate initiator (Sigma). Following purification and drying, the resulting PLA-PEG-biotin was dissolved at 1 mg/ml in trifluoroethanol (TFE) and 1 ml was cast into each well of a 6-well plate and allowed to evaporate overnight to form thin films. The film is composed of a degradable block copolymer (23,400 Mw) that presents pegylated biotin groups to the aqueous phase. The PEG chain acts as a flexible linker reducing steric hindrance and enhances the ability of the biotin unit to bind the tetrameric protein avidin (Salem et al., 2001).

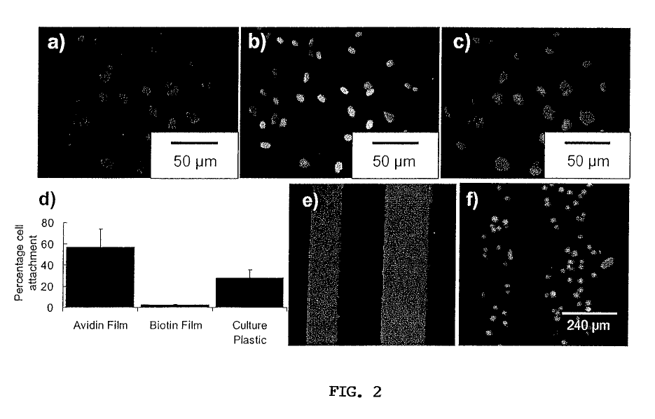

The avidin was patterned on the PLA-PEG-biotin using a PDMS stamp with channels ranging from 100 μm to 250 μm in width. The PDMS mold was placed onto the film and 1 ml of a 500 μg/ml solution of tetramethylrhodamine conjugated avidin (av-rh, Molecular probes) in d.i. water was placed so that it wetted the PDMS molds capillary entrances. After 1 hr of contact, the remaining av-rh was removed by blotting and replaced with 5 ml of distilled water. After a further 5 min, the water was removed and the washing procedure repeated another 5 times. The sample was then immersed in water and the mold removed by carefully peeling apart the PLA-PEG-biotin substrate and the PDMS. The sample was then washed several times with an additional 50 mL of water. The patterned immobilized avidin as illustrated in FIGS. 2A-F provides spatially defined binding sites for biotinylated cells to bind to.

Human Dermal Fibroblasts (HDF) were selected as a model cell line for biotinylation based on previous reports that have shown the importance of spatial control over fibroblast attachment in maintaining viability of co-cultures (Bhatia et al., 1998). To prove that the inventors could biotinylate the HDF cells, they were grown to 65-70% confluence in 12-well plates. Cell culture media in the wells was removed. Celltracker Red™ (Molecular Probes) that had been reconstituted in 11 μl of dimethylsiloxane (DMSO) and mixed into media warmed to 37° C. was added to the wells and incubated for 45 minutes at 37° C., 5% CO₂. The media was then removed and replaced with fresh warmed media and incubated for a further 1 hr. The cells were washed twice with phosphate buffered saline (PBS) and incubated with a 1 mM solution of sodium periodate in cold PBS for 15 minutes in the dark at 4° C. The HDF cells were then washed with buffer 1 (PBS, 0.1% Bovine Calf Serum, pH 6.5) at room temperature and then incubated with a 5 mM solution of biotin hydrazide (Sigma) in buffer 1 for 90 min at room temperature. Cells were then washed twice in buffer 2 (PBS, 0.1% BCS, pH 7.4) and incubated with a 5 μg/ml of FITC-avidin (Molecular Probes) in buffer 2 solution for 15 minutes at 4° C. The samples were incubated with a 0.01 mg/ml solution of Hoechst 33258 (Aldrich) in PBS. All the cells were then washed twice in buffer 2 prior to imaging by fluorescent microscopy (Olympus BX40). FIG. 2A shows an image of celltracker red stained cells with the corresponding images showing the same cells stained with hoescht (FIG. 2B) and fluorescence (FIG. 2C) emanating from the FITC labeled avidin that has bound specifically to the biotinylated cells. The degree of biotinylation was calculated to be 1.9±0.19×10⁹ biotin moieties per cell. Control experiments in which the cells were treated with every step except the periodate treatment and control experiments in which the cells were treated with every step except for the biotin functionalization did not show any fluorescence through the FITC channel confirming that the fluorescence observed was due to a specific avidin-biotin receptor mediated interaction. The biotin-avidin interaction has been reported to primarily increase cell attachment in the first hour (Bhat et al., 1998c; Tsai & Wang, 2005). Reverse transcriptase-polymerase chain reaction (RT-PCR) analysis of the long-term culture of another cell type (chondrocytes) has shown that this exceptionally strong biological binding interaction (Kₐ=10⁻¹⁵ M⁻¹) does not interfere with the cells long-term ability to proliferate or produce extracellular matrix proteins in comparison to untreated cells (Tsai & Wang, 2005).

Cell viability measurements were carried out using trypan blue measurements. Biotin functionalized cells demonstrated an 88.3% viability in comparison to a 95.33% for untreated cells.

To evaluate the binding affinity of biotinylated cells with an avidin immobilized PLA-PEG-biotin substrate, the biotinylated cells were washed with 5 mM EDTA and then trypsinized and washed. 5×10⁵ cells were added in serum free medium to PLA-PEG-biotin films in each well of a 6-well plate that had been saturated with avidin (Sigma) and washed 3 times with PBS. Cultures were allowed to attach for 15 min before washing with PBS. Attached cells were observed by light microscopy. No difference in viability was observed after exposure to the avidin coated polymer substrate. As illustrated in FIG. 2D, biotinylated cell attachment observed on the avidinylated substrates was 25-fold higher than biotinylated cell attachment on non-avidin coated PLA-PEG-biotin substrates and 2-fold higher than TCP. When adherent cells were washed with avidin made up to 5×10⁻⁷ M in dibasic phosphate buffer (10 mM, pH 7.4), the binding process was not reversed.

Next, HDF cells grown to 65-70% confluence in T75 flasks were incubated for 45 min at 37° C., 5% CO₂ with Celltracker Green™ (Molecular Probes) that had been reconstituted in 11 μl of DMSO and mixed into media warmed to 37° C. The HDF cells were biotinylated as described earlier and then 5×10⁵ cells in serum free media were added onto the PLA-PEG-biotin substrates that had been patterned with av-rh. After 15 min of incubation with the cells, the patterned templates were washed with PBS. Attached cells were observed by fluorescent microscopy. As illustrated in FIG. 2F, cells labeled with celltracker green were found to be bound specifically within the patterned avidin lanes. Cell binding was minimized outside the avidin patterned channels by PEG presented from the copolymer. Additional experiments, utilizing non-adherent Raji, Daudi and Jurkat cells were successfully performed as well.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:
1. Tien J, Chen C S. Patterning the cellular microenvironment. Ieee Engineering in Medicine and Biology Magazine 2002;21(1):95-98.
2. Hyun J H, Ma H W, Zhang Z P, Beebe T P, Chilkoti A. Universal route to cell micropatterning using an amphiphilic comb polymer. Advanced Materials 2003;15(7-8):576-579.
3. Raghavan S, Chen C S. Micropatterned environments in cell biology. Advanced Materials 2004;16(15):1303-1313.
4. Patel N, Padera R, Sanders G H W, Cannizzaro S M, Davies M C, Langer R, et al. Spatially controlled cell engineering on biodegradable polymer surfaces. Faseb Journal 1998;12(14):1447-1454.
5. TessierLavigne M, Goodman C S. The molecular biology of axon guidance. Science 1996;274(5290):1123-1133.
6. Bhatia S N, Balis U J, Yarmush M L, Toner M. Microfabrication of hepatocyte/fibroblast co-cultures: Role of homotypic cell interactions. Biotechnology Progress 1998;14(3):378-387.
7. Chen C S, Jiang X Y, Whitesides G M. Microengineering the environment of mammalian cells in culture. Mrs Bulletin 2005;30(3):194-201.
8. Kane R S, Takayama S, Ostuni E, Ingber D E, Whitesides G M. Patterning proteins and cells using soft lithography. Biomaterials 1999;20(23-24):2363-2376.
9. Chen C S, Mrksich M, Huang S, Whitesides G M, Ingber D E. Geometric control of cell life and death. Science 1997;276(5317):1425-1428.
10. Tan W, Desai T A. Microfluidic patterning of cells in extracellular matrix biopolymers: Effects of channel size, cell type, and matrix composition on pattern integrity. Tissue Engineering 2003;9(2):255-267.
11. Tourovskaia A, Figueroa-Masot X, Folch A. Differentiation-on-a-chip: A microfluidic platform for long-term cell culture studies. Lab on a Chip 2005;5(1):14-19.
12. Patel N, Bhandari R, Shakesheff K M, Cannizzaro S M, Davies M C, Langer R, et al. Printing patterns of biospecifically-adsorbed protein. Journal of Biomaterials Science-Polymer Edition 2000; 11(3):319-331.
13. Mrksich M, Dike L E, Tien J, Ingber D E, Whitesides G M. Using microcontact printing to pattern the attachment of mammalian cells to self-assembled monolayers of alkanethiolates on transparent films of gold and silver. Experimental Cell Research 1997;235(2):305-313.
14. Lin C C, Co C C, Ho C C. Micropatterning proteins and cells on polylactic acid and poly(lactide-co-glycolide). Biomaterials 2005;26(17):3655-3662.
15. Liu V A, Jastromb W E, Bhatia S N. Engineering protein and cell adhesivity using PEO-terminated triblock polymers. Journal of Biomedical Materials Research 2002;60(1):126-134.
16. Bhat V D, Klitzman B, Koger K, Truskey G A, Reichert W M. Improving endothelial cell adhesion to vascular graft surfaces: Clinical need and strategies. Journal of Biomaterials Science-Polymer Edition 1998;9(11):1117-1135.
17. Bhat V D, Truskey G A, Reichert W M. Fibronectin and avidin-biotin as a heterogeneous ligand system for enhanced endothelial cell adhesion. Journal of Biomedical Materials Research 1998;41(3):377-385.
18. Bhat V D, Truskey G A, Reichert W M. Using avidin-mediated binding to enhance initial endothelial cell attachment and spreading. Journal of Biomedical Materials Research 1998;40(1):57-65.
19. Tsai W B, Wang M C. Effect of an avidin-biotin binding system on chondrocyte adhesion, growth and gene expression. Biomaterials 2005;26(16):3141-3151.
20. Miyata T, Conte M S, Trudell L A, Mason D, Whittemore A D, Birinyi L K. Delayed Exposure to Pulsatile Shear-Stress Improves Retention of Human Saphenous-Vein Endothelial-Cells on Seeded Eptfe Grafts. Journal of Surgical Research 1991;50(5):485-493.
21. Prescher J A, Dube D H, Bertozzi C R. Chemical remodelling of cell surfaces in living animals. Nature 2004;430(7002):873-877.
22. Luchansky S J, Goon S, Bertozzi C R. Expanding the diversity of unnatural cell-surface sialic acids. Chembiochem 2004;5(3):371-374.
23. De Bank P A, Kellam B, Kendall D A, Shakesheff K M. Surface engineering of living myoblasts via selective periodate oxidation. Biotechnology and Bioengineering 2003;81(7):800-808.
24. Salem A K, Cannizzaro S M, Davies M C, Tendler S J B, Roberts C J, Williams PM, et al. Synthesis and characterisation of a degradable poly(lactic acid)-poly(ethylene glycol) copolymer with biotinylated end groups. Biomacromolecules 2001;2(2):575-580.
25. Salem A K, Chao J, Leong K W, Searson P C. Receptor-mediated self-assembly of multi-component magnetic nanowires. Advanced Materials 2004;16(3):268-271.
26. Salem A K, Rose F, Oreffo R O C, Yang X B, Davies M C, Mitchell J R, et al. Porous polymer and cell composites that self-assemble in situ. Advanced Materials 2003;15(3):210-213.
Luong et al., in Practical Fluorescence, G. Guilbault ed., Marcel Dekker, New York, 1990.
Hughes et al., Analytica Chimica Acta 307:393, 1995.
Koop et al., Biochem. J. 295:165, 1993.
Gura, Science 276:1989, 1997.
Niswender et al., J. Microscopy 180(2): 109, 1995.
Cubitt et al., TIBS 20:448, 1995.
Miyawaki et al., Nature 388:882, 1997.
Cobbold et al., Cell Biology 1:311, 1990.
U.S. Pat. No. 6,856,125
U.S. Pat. No. 6,667,159
U.S. Pat. No. 6,605,039
U.S. Pat. No. 6,603,548
U.S. Pat. No. 6,577,780
U.S. Pat. No. 6,544,393
U.S. Pat. No. 6,377,721
U.S. Pat. No. 6,329,160
U.S. Pat. No. 6,274,345
U.S. Pat. No. 6,258,254
U.S. Pat. No. 6,210,910
U.S. Pat. No. 5,177,012
ATCC Cell Lines and Hybridomas, 8th ed., 1994.
Bacteria and Bacteriophages, 19th ed., 1996.
Yeast, 1995.
Mycology and Botany, 19th ed., 1996.
Protists: Algae and Protozoa, 18th ed., 1993.

What is claimed is:

1. A method of immobilizing cells comprising:
(a) providing a surface comprising avidin, wherein in avidin is disposed in a pre-determined pattern; and
(b) contacting said surface comprising avidin with a biotinylated cell population,
whereby said cell population is immobilized to said surface according to said predetermined pattern.

2. The method of claim 1, wherein said avidin is attached to said surface by interaction with biotin bound to said surface.

3. The method of claim 2, wherein said biotin is bound to said surface through a block copolymer, which is in turn bound to said surface.

4. The method of claim 3, wherein said block copolymer is biodegradable.

5. The method of claim 2, wherein said block copolymer is silk, elastin, chitin, chitosan, poly(d-hydroxy acid), poly(anhydrides), and poly(athoesters).

6. The method of claim 3, further comprising a step, prior to step (a), of applying said block copolymer to said surface.

7. The method of claim 1, further comprising a step, prior to step (a), of applying said avidin to said surface.

8. The method of claim 7, wherein applying avidin to said surface comprises microfluidic networking.

9. The method of claim 8, wherein said microfluidic networking employs a poly-dimethylsiloxane stamp.

10. The method of claim 1, further comprising a step, prior to step (b), of biotinylating said cell.

11. The method of claim 10, wherein biotinylating said cell comprises treating said cell with sodium periodate to generate a cell comprising non-native aldehydes, and reacting said cell comprising non-native aldehydes with biotin-hydrazine.

12. The method of claim 1, wherein said surface further comprises block copolymer lacking avidin in areas outside said predetermined pattern.

13. The method of claim 12, wherein said block copolymer lacking avidin is biodegradable.

14. The method of claim 12, wherein said cell population is bound to said predetermined pattern at levels about 10-fold, about 15-fold, about 20-fold or about 25-fold higher than outside said predetermined pattern.

15. The method of claim 1, wherein said cell population is naturally adherent.

16. The method of claim 1, wherein said cell population is naturally non-adherent.

17. The method of claim 1, wherein said cell population is a fibroblast population, an endothelial cell population, a neuronal cell population, or an epithelial cell population.

18. The method of claim 1, wherein said surface comprises silicon, plastic, glass or paper.

19. The method of claim 1, wherein said surface is disposed in a culture dish, a biochip, a column, or a filter.

20. The method of claim 1, wherein said predetermined pattern comprises cells dispersed in a line, a square, a rectangle, a circle, an oval or combinations thereof.

21. The method of claim 1, wherein said cell population, when bound to said surface, is 85% viable.

22. A device comprising:
(a) a surface;
(b) a biotinylated copolymer bound to said surface;
(c) avidin bound to said biotinylated copolymer in a predetermined pattern; and
(d) a biotinylated cell population bound to said avidin.
